(19) United States
(12) Patent Application Publication
(10) Pub. No.: US 2021/0361677 A1
(43) Pub. Date: Nov. 25, 2021

(54) DOSING METHODS FOR TREATING INFLAMMATORY BOWEL CONDITIONS

(71) Applicant: Intact Therapeutics, Inc., Belmont, CA (US)

(72) Inventors: Ravinder D. Pamnani, Los Altos, CA (US); Sidhartha Ranjit Sinha, San Francisco, CA (US); Karolin Julia Jarr, Burg Stargard (DE); Navdeep Kaur, Fremont, CA (US)

(21) Appl. No.: 17/324,316

(22) Filed: May 19, 2021

Related U.S. Application Data
(60) Provisional application No. 63/028,771, filed on May 22, 2020.

(51) Int. Cl. A61K 31/606; A61K 47/10; A61P 1/00; A61K 9/00

(52) U.S. Cl. CPC A61K 31/606; A61K 9/0031; A61P 1/00; A61K 47/10

(57) ABSTRACT

The invention provides methods and compositions for local administration of therapeutic agents to the rectum or colon, such as by enema. The methods and compositions are useful for treatment of inflammatory bowel disease, including Crohn's disease and ulcerative colitis.

### CROSS-REFERENCE TO RELATED APPLICATIONS

This application claims the benefit of, and priority to, U.S. Provisional Patent Application No. 63/028,771, filed May 22, 2020, the contents of which are incorporated by reference.

### FIELD OF THE INVENTION

The invention generally relates to dosing methods for treating conditions of the large bowel, such as inflammatory bowel disease and irritable bowel syndrome.

### BACKGROUND

Inflammatory bowel disease (IBD) afflicts about 3 million Americans and over 11 million people worldwide. IBD is a group of inflammatory conditions of the gastrointestinal tract, including the mouth, esophagus, stomach, small intestine, and colon, with Crohn's disease and ulcerative colitis being the predominant forms. IBD causes debilitating symptoms, such as abdominal pain, diarrhea, rectal bleeding, cramping, weight loss, and anemia, and can be fatal if left untreated.

Anti-inflammatory agents, such as mesalamine (also called mesalazine or 5-aminosalicylic acid (5-ASA)), or corticosteroids, such as budesonide, are often used to treat IBD. However, despite the existence of suitable pharmacological agents to combat many forms of IBD, treatment of IBD is hampered by the difficulties of administering such agents. For example, when drugs such as mesalamine or budesonide are administered orally, most of the active agent is metabolized as it passes through the gastrointestinal tract or eliminated through bulk transit. Consequently, oral formulations generally fail to achieve therapeutic concentrations of the drug in the distal colon, the site of lesions in ulcerative colitis and many cases of Crohn's disease. Formulations designed to overcome this obstacle by preventing release of the drug in the stomach are hindered by other problems, such as incomplete release, release in the proximal rather than distal colon, release of toxic metabolites, and high patient-to-patient variability. Another concern is that oral or parenteral administration of immunosuppressive agents increases the risk of malignancies and infections.

Current methods of local administration are plagued by their own set of problems. The GI tract, including the colon, is designed to continuously move consumed content through the body while absorbing nutrients. As a result, therapeutic agents administered locally, i.e., directly to the surface of the colon, tend to get rapidly cleared from target tissue during transit of digested material through the bowel. Because treatment of inflamed or infected GI tissue requires sustained exposure of such tissue to therapeutic agents, frequent administration (e.g., daily or multiple times per day) is often necessary to achieve the full therapeutic benefit of a locally-administered agent. As a result, many patients fail to comply with a prescribed dosing regimen.

Other barriers to effective local treatment of IBD stem from the mode of administration. Enemas permit delivery of therapeutic agents to the entirety of the descending colon. However, enema-based treatments typically require patients to retain a substantial volume of liquid (e.g., 60-100 ml) in the colon for an extended period in multiple daily administrations, an unpleasant exercise that further hampers patient compliance. Suppositories and foams are less inconvenient than enemas but generally fail to deliver agents beyond the rectum and sigmoid colon, respectively. One of the primary symptoms of IBD is urgency and diarrhea, and a bowel movement will clear out the enema, foam, or suppository formulations, thereby limiting efficacy. Consequently, existing methods for delivering therapeutic agents are inadequate to treat many forms of IBD, and millions of people continue to suffer from conditions such as Crohn's disease and ulcerative colitis.

### SUMMARY

The invention provides dosing methods for treating conditions of the gastrointestinal tract, such as inflammatory bowel conditions. Aspects of the invention are accomplished using a formulation that exists as a liquid initially but forms a gel when a condition of the formulation exceeds a threshold level. The condition may be physical, e.g., temperature, chemical, e.g., pH, temporal, or any combination of those conditions. For example, in certain methods of the invention, the formulation exists as a liquid at room temperature, e.g., about 23° C., and transitions to a gel at or near human body temperature, about 37° C. The invention leverages such formulations to dose active agents locally.

In certain aspects, the methods of the invention involve administering a therapeutic agent locally to the colon, e.g., by enema, to achieve prolonged retention of the agent and sustained therapeutic effect. It has now been discovered that because the methods afford superior retention of the agent in the colon, they decrease the frequency at which enemas must be administered. For example, data herein shows that methods of the invention enable patients to go three days or more between administrations, leading to higher rates of patient compliance. In addition, the methods allow delivery of agents to the entirety of the left colon. Thus, the methods are useful for treating IBD, including Crohn's disease and ulcerative colitis.

The invention also recognizes that, due to the prolonged intervals between dosages, methods of the invention are effective for different phases of treatment of IBD. Treatment of IBD typically entails an initial phase to induce remission of the condition (induction phase) and a subsequent, ongoing phase to maintain remission (maintenance phase), with the induction phase requiring higher dosage and/or frequency of administration. By adjusting either or both of these variables, methods of the invention allow the same agent to be used for both induction and maintenance phases of therapy. Moreover, in contrast to prior enema-based methods of administration, the present methods allow sufficient intervals between dosages to make enematic delivery practical for long-term therapeutic regimens associated with maintenance phase.

Data herein further show that the methods also improve therapeutic efficacy by providing sustained exposure of inflamed tissue to the therapeutic agent. Thus, certain methods obviate the need for multiple daily administrations for many therapeutic applications.

Another interesting discovery is that the methods allow therapeutic benefits to endure following a bowel movement, obviating the need for patients to face the unpleasant choice of either refraining from defecating or repeating the enema procedure.

Specific examples of the novel dosing methods are now provided. In an aspect, the invention provides methods of treating a gastrointestinal condition in a subject by providing an agent locally to the rectum or colon of a subject according to a first dosing regimen and providing the agent locally to the rectum or colon of the subject according to a second dosing regimen that is different from the first dosing regimen.

In another aspect, the invention provides methods of maintaining a gastrointestinal condition in a reduced state in a subject by providing an agent locally to the rectum or colon of a subject, wherein the agent is provided following an acute phase of the condition, thereby maintaining the gastrointestinal condition in a reduced state. The subject may have been previously treated for the condition. The previous treatment may have induced remission of the condition. The previous treatment may include providing the agent to the subject. The previous treatment may include providing the agent locally to the rectum or colon of the subject. The previous treatment may include providing the agent systemically to the rectum or colon of the subject.

In another aspect, the invention provides methods of treating a gastrointestinal condition in a subject by providing an agent locally to the rectum or colon of a subject according to a dosing regimen comprising a dosing interval that is greater than 12 hours, wherein the therapeutic effect of the agent is maintained during the dosing interval.

In another aspect, the invention provides methods of treating a gastrointestinal condition in a subject by providing an agent locally to the rectum or colon of a subject, wherein the method is not repeated following a bowel movement by the subject and a therapeutic effect of the agent is maintained after the bowel movement without the method being repeated.

For the above described methods, the agent may be provided topically, such as rectally. In certain embodiments, the agent may be provided by enema, as a suppository, or as a foam. In certain embodiments, providing the agent may induce remission of the condition in the subject. In other embodiments, providing the agent may maintain remission of the condition in the subject.

In methods that include two dosing regimens, providing the agent according to the first dosing regimen may induce remission of the condition in the subject. In methods that include two dosing regimens, providing the agent according to the second dosing regimen may maintain remission of the condition in the subject. In methods that include two dosing regimens, the dosing interval of the first dosing regimen may be the less than, the same as, or greater than the dosing interval of the second dosing regimen. In methods that include two dosing regimens, the dosage of the first dosing regimen may be the less than, the same as, or greater than the dosage of the second dosing regimen.

In methods that include two dosing regimens, the agent may be provided by the same route in each dosing regimen. The agent may be provided rectally in both the first and second dosing regimens. Alternatively, in methods that include two dosing regimens, the agent may be provided by a different route in each dosing regimen. The agent may be provided rectally in the first dosing regimen and by another route in the second dosing regimen. The agent may be provided rectally in the second dosing regimen and by another route in the first dosing regimen.

Each dosing regimen may include a dosage, a dosing interval, or both. Each dosing interval may independently be about 6 hours, about 8 hours, about 12 hours, about 15 hours, about 24 hours, about 36 hours, about 48 hours, about 60 hours, about 72 hours, about 84 hours, about 96 hours, about 5 days, about 6 days, about 7 days, about 8 days, about 10 days, about 12 days, about 14 days, about 3 weeks, about 4 weeks, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 10 days, at least 12 days, at least 14 days, at least 3 weeks, at least 4 weeks, greater than 6 hours, greater than 8 hours, greater than 12 hours, greater than 15 hours, greater than 24 hours, greater than 36 hours, greater than 48 hours, greater than 60 hours, greater than 72 hours, greater than 84 hours, greater than 96 hours, greater than 5 days, greater than 6 days, greater than 7 days, greater than 8 days, greater than 10 days, greater than 12 days, greater than 14 days, greater than 3 weeks, or greater than 4 weeks.

The agent may be any agent that provides a therapeutic benefit when administered to the rectum or colon of a subject.

The agent may be an aminosalicylate, angiotensin receptor inhibitor, anti-inflammatory, antibiotic, antibody, antimetabolite, antimycotic, antisense oligonucleotide, bacterial sample, beta-blocker, biologic, budesonide, calcineurin inhibitor, corticosteroid, cytokine, growth factor, immunosuppressor, indole, interferon, Janus kinase inhibitor, microbial metabolite, mTOR inhibitor, opioid, PDE4 inhibitor, peptide, pregnane X receptor (PXR) ligand, probiotic, protein, proton pump inhibitor (PPI), salicylic acid derivative, small molecule, sphingosine-1-phosphate receptor modulator, thiopurine, TNF-α binding protein, or toll-like receptor (TLR) ligand.

The aminosalicylate may be 5-ASA, 4-ASA, azodisalicylate, balsalazide, ipsalazide, olsalazine, or sulfasalazine.

The angiotensin receptor inhibitor may be candesartan, eprosartan, fimasartan, irbesartan, losartan, olmesartan, saprisartan, telmisartan, orvalsartan.

The antimycotic may be abafungin, albaconazole, amorolfin, amphotericin B, anidulafungin, bifonazole, butenafine, butoconazole, candicidin, caspofungin, clotrimazole, econazole, efinaconazole, epoxiconazole, fenticonazole, filipin, fluconazole, hamycin, isavuconazole, isoconazole, itraconazole, ketoconazole, luliconazole, micafungin, miconazole, naftifine, natamycin, nystatin, omoconazole, oxiconazole, posaconazole, propiconazole, ravuconazole, rimocidin, sertaconazole, sulconazole, terbinafine, terconazole, tioconazole, or voriconazole.

The beta blocker may be acebutolol, atenolol, betaxolol, bisoprolol, bucindolol, butaxamine, carteolol, carvedilol, celiprolol, esmolol, ICI-118,551, labetalol, metoprolol, nadolol, nebivolol, oxprenolol, penbutolol, pindolol, propanolol, sotalol, SR 59230A, or timolol.

The biologic may be a TNF-alpha inhibitor, such as adalimumab, certolizumab pegol, golimumab, or infliximab; an integrin receptor antagonist, such as natalizumab or vedolizumab; or an interleukin antagonist, such as ustekinumab. The biologic may be a biosimilar of another biologic.

The calcineurin inhibitor may be ciclosporin, pimecrolimus, or tacrolimus.

The corticosteroid may be beclamethasone, budesonide, dexamethasone, prednisolone, or prednisone.

The immunosuppressor may be 6-mercaptopurine, 6-thioguanine, azathioprine, cyclosporine, methotrexate, mycophenolate, or tacrolimus.

The indole may be indole, indole 3 acetic acid (IAA), or indole 3 propionic acid (IPA).

The interferon may be interferon alfa-2a, interferon alfa-2b, interferon alfacon-1, interferon alfa-n3, interferon beta-1a, interferon beta-1b, interferon gamma-1b, peginterferon alfa-2a, peginterferon alfa-2b, peginterferon alfa-2b, or peginterferon beta-1a.

The Janus kinase (JAK) inhibitor may be filgotinib, peficitinib, tofacitinib, upadicitinib, Pf-06651600, Pf-06700841, or TD-1473.

The microbial metabolite may be indole, indole 3 acetic acid (IAA), or indole 3 propionic acid (IPA).

The mTOR inhibitor may be everolimus, sirolimus, or temsirolimus.

The opioid may be alfentanil, carfentanil, etorphine, fentanyl, hydromorphone, morphine, pethidine, remifentanil, or sufentanil.

The PDE4 inhibitor may be apremilast, cilomilast, crisaborole diazepam, ibudilast, luteolin, mesembrenone, piclamilast, roflumilast, or rolipram.

The pregnane X receptor (PXR) ligand may be BAS451, FKK1, FKK2, FKK3, FKK4, FKK5, FKK6, FKK7, FKK8, FKK9, FKK10, FKK999, hyperforin, rifampicin, rifaximin, or SR12813.

The proton pump inhibitor (PPI) may be dexlansoprazole, esomeprazole, lansoprazole, omeprazole, pantoprazole, or rabeprazole.

The salicylic acid derivative may be 5-aminosalicylic acid, or a derivate/variant, such as 4-aminosalicylic acid, azodisalicylate, balsalazide, ipsalazide, olsalazine, or sulfasalazine.

The sphingosine-1-phosphate receptor modulator may be fingolimod, laquinimod, ozanimod, ponesimod, or siponimod.

The thiopurine may be 6-mercaptopurine (6-MP), 6-thioguanine (6-TG), or azathioprine (AZA).

The TNF-α binding protein may be adalimumab, certolizumab pegol, etanercept, golimumab, or infliximab.

The TLR ligand may be 3-deoxy-D-manno-octulosonic acid (KDO2)-lipid A.

The agent may be provided in a formulation that exists as a liquid when the formulation is below a threshold condition and as a gel when the formulation is above threshold condition. The threshold may be any combination of physical, chemical, and temporal conditions.

The physical condition may be temperature. The threshold condition may be a transition temperature. The formulation may exist as a liquid when the formulation is below the transition temperature and as a gel when the formulation is above the transition temperature. The formulation may exist as a liquid when the formulation is above the transition temperature and as a gel when the formulation is below the transition temperature.

The chemical condition may be acidity, alkalinity, or pH. The threshold condition may be a transition pH. The formulation may exist as a liquid when the formulation is below the transition pH and as a gel when the formulation is above the transition pH. The threshold condition may be a transition pH. The formulation may exist as a liquid when the formulation is above the transition pH and as a gel when the formulation is below the transition pH.

The temporal condition may be time. The threshold condition may be a transition time point. The formulation may exist as a liquid prior to the transition time point and as a gel after the transition time point.

The agent may be provided in a formulation that exists as a liquid at a first temperature and transitions to a gel at a second temperature. The first temperature may be lower than or higher than the second temperature. The first temperature may be about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C. The second temperature may be about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. The formulation may transition to a gel at from about 16° C. to about 40° C., from about 18° C. to about 40° C., from about 20° C. to about 40° C., from about 22° C. to about 40° C., from about 24° C. to about 40° C., from about 26° C. to about 40° C., from about 28° C. to about 40° C., from about 30° C. to about 40° C., from about 32° C. to about 40° C., from about 34° C. to about 40° C., from about 36° C. to about 40° C., from about 38° C. to about 40° C., from about 16° C. to about 38° C., from about 18° C. to about 38° C., from about 20° C. to about 38° C., from about 22° C. to about 38° C., from about 24° C. to about 38° C., from about 26° C. to about 38° C., from about 28° C. to about 38° C., from about 30° C. to about 38° C., from about 32° C. to about 38° C., from about 34° C. to about 38° C., from about 36° C. to about 38° C., from about 16° C. to about 36° C., from about 18° C. to about 36° C., from about 20° C. to about 36° C., from about 22° C. to about 36° C., from about 24° C. to about 36° C., from about 26° C. to about 36° C., from about 28° C. to about 36° C., from about 30° C. to about 36° C., from about 32° C. to about 36° C., from about 34° C. to about 36° C., from about 16° C. to about 34° C., from about 18° C. to about 34° C., from about 20° C. to about 34° C., from about 22° C. to about 34° C., from about 24° C. to about 34° C., from about 26° C. to about 34° C., from about 28° C. to about 34° C., from about 30° C. to about 34° C., from about 32° C. to about 34° C., from about 16° C. to about 32° C., from about 18° C. to about 32° C., from about 20° C. to about 32° C., from about 22° C. to about 32° C., from about 24° C. to about 32° C., from about 26° C. to about 32° C., from about 28° C. to about 32° C., from about 30° C. to about 32° C., from about 16° C. to about 30° C., from about 18° C. to about 30° C., from about 20° C. to about 30° C., from about 22° C. to about 30° C., from about 24° C. to about 30° C., from about 26° C. to about 30° C., from about 28° C. to about 30° C., from about 16° C. to about 28° C., from about 18° C. to about 28° C., from about 20° C. to about 28° C., from about 22° C. to about 28° C., from about 24° C. to about 28° C., from about 26° C. to about 28° C., from about 16° C. to about 26° C., from about 18° C. to about 26° C., from about 20° C. to about 26° C., from about 22° C. to about 26° C., from about 24° C. to about 26° C., from about 16° C. to about 24° C., from about 18° C. to about 24° C., from about 20° C. to about 24° C., from about 22° C. to about 24° C., from about 16° C. to about 22° C., from about 18° C. to about 22° C., from about 20° C. to about 22° C., from about 16° C. to about 20° C., or from about 18° C. to about 20° C.

The formulation may include the agent in a mixture with a polymer. The polymer may be a block copolymer. The block copolymer may include one or more of polyethylene glycol and polypropylene glycol.

The formulation may include a lipid. The lipid may be a fatty acid, glycolipid, phosphoglyceride, phospholipid, sphingolipid, or sterol. The lipid may be glucosyl-cerebroside, phosphatidylcholine, phosphatidylethanolamine, phosphatidylinositol, phosphatidylserine, sphingomyelin.

The gastrointestinal condition may be achalasia, Barrett's esophagus, Boerhaave syndrome, celiac disease, constipation, Crohn's disease, diverticulitis, enteritis, enterocolitis, eosinophilic esophagitis, esophageal burns, esophageal candidiasis, esophageal spasm, esophageal stricture, esophageal webbing, esophageal varices, esophagitis, gastritis, gastritis, gastroenteritis, gastroesophageal reflux disease, gastrointestinal bleeding, indeterminate colitis, inflammatory bowel disease, intestinal graft-versus-host disease, irritable bowel syndrome, Mallory-Weiss tears, microscopic colitis, nutcracker esophagus, oral mucositis, pernicious anemia, pouchitis, radiation colitis, radiation esophagitis, radiation proctitis, ulcerative colitis, ulcers, or Zenker's diverticulum.

The agent may be retained in the colon for a defined period. The agent may be retained in the colon for at least 6 hours, at least 8 hours, at least 12 hours, at least 15 hours, at least 24 hours, at least 36 hours, at least 48 hours, at least 60 hours, at least 72 hours, at least 4 days, at least 5 days, at least 7 days, greater than 6 hours, greater than 8 hours, greater than 12 hours, greater than 15 hours, greater than 24 hours, greater than 36 hours, greater than 48 hours, greater than 60 hours, greater than 72 hours, greater than 4 days, greater than 5 days, or greater than 7 days.

The agent may maintain a therapeutic effect in the colon for a defined period. The agent may maintain a therapeutic effect in the colon for at least 6 hours, at least 8 hours, at least 12 hours, at least 15 hours, at least 24 hours, at least 36 hours, at least 48 hours, at least 60 hours, at least 72 hours, at least 4 days, at least 5 days, at least 7 days, greater than 6 hours, greater than 8 hours, greater than 12 hours, greater than 15 hours, greater than 24 hours, greater than 36 hours, greater than 48 hours, greater than 60 hours, greater than 72 hours, greater than 4 days, greater than 5 days, or greater than 7 days.

The method may include systemic administration of the agent to the subject. The method may not include systemic administration of the agent to the subject.

### BRIEF DESCRIPTION OF THE DRAWINGS

FIG. 1 is graph showing plasma levels of 5-ASA in two patients following administration by enema according to a prior art method.

FIG. 2 is a schematic of the randomized double-blind, placebo-controlled crossover study design used to assess thermosensitive delivery platform (TDP) versus liquid control enema.

FIG. 3 is a table showing the age and gender of subjects who participated in the study to assess thermosensitive delivery platform (TDP) versus liquid control enema.

FIG. 4 is a graph showing the adverse effects of TDP compared to a liquid control.

FIG. 5 is a graph showing intolerability of liquid control versus TDP.

FIG. 6 is a table showing reference of TDP versus liquid control enema.

FIG. 7 is a graph showing the maximum proximal distribution of enema after instillation as assessed by x-ray.

FIG. 8 is a table showing the change in proximal distribution over time of TDP and control enemas.

FIG. 9 is a graph showing the change between maximum proximal distance achieved by TDP versus control enema at t=0 hour versus t=6 hour.

FIG. 10 shows abdominal x-ray images after control enema at t=0 hour versus t=6 hour.

FIG. 11 shows abdominal x-ray images TDP enema at t=0 hour versus t=6 hour.

FIG. 12 shows endoscopic images after control enema.

FIG. 13 shows endoscopic images after TDP enema.

FIG. 14 is a table showing plasma detection of TDP using ultraviolet-visible spectrophotometry.

FIG. 15 shows mucus layer staining of colon biopsies. Arrow highlights mucin 2 protein staining.

### DETAILED DESCRIPTION

The invention provides methods of treating gastrointestinal (GI) conditions by providing an agent locally to the colon of a subject. The methods are useful for treating inflammatory bowel disease (IBD)

Prior methods of administering therapeutic agents to the colon are beset with problems. Agents such as 5-aminosalicylic acid (5-ASA) that treat IBD lesions in the colon act locally, rather than systemically, so they are most effective when delivered directly to affected tissue. One shortcoming of conventional oral formulations of such therapeutic agents is that that the active agent is metabolized and/or eliminated as it passes through the GI tract. Consequently, therapeutically effective concentrations of the agent are seldom achieved in the distal colon, which is often the site of lesions. See, e.g., De Vos, et al., Concentrations of 5-ASA and Ac-5-ASA in human ileocolonic biopsy homogenates after oral 5-ASA preparations, Gut. 1992 October; 33(10):1338-42, the contents of which are incorporated herein by reference. Enterically-coated formulations are imperfect solutions because they suffer from issues such as incomplete release of the active agent, release in the proximal rather than distal colon, and high patient-to-patient variability. See, e.g., Ghosh and Daperno, Topical therapy in ulcerative colitis: always a bridesmaid but never a bride? Gastroenterology, 2015 April; 148(4):701-4. doi: 10.1053/j.gastro.2015.02.038; Karkossa and Klein, A Biopredictive In Vitro Comparison of Oral Locally Acting Mesalazine Formulations by a Novel Dissolution Model for Assessing Intraluminal Drug Release in Individual Subjects, J Pharm Sci. 2018 June; 107(6):1680-1689. doi: 10.1016/j.xphs.2018.02.016; and Maroni, et al., Enteric coatings for colonic drug delivery: state of the art, Expert Opin Drug Deliv. 2017 September; 14(9):1027-1029. doi: 10.1080/17425247.2017.1360864, the contents of each of which are incorporated herein by reference. Likewise, suppositories and foams generally fail to deliver adequate levels of therapeutic agent to the site of lesions because their zone of exposure is limited to the rectum. See, e.g., Brunner, et al., Colonic spread and serum pharmacokinetics of budesonide foam in patients with mildly to moderately active ulcerative colitis, Aliment Pharmacol Ther 2005; 22: 463-470, doi: 10.1111/j.1365-2036.2005.02571.x, the contents of which are incorporated herein by reference. The only prior methods that provide adequate doses of therapeutic agents to the colon are enema, but such methods have their own limitations. One is that most of the therapeutic agent is eliminated along with fecal matter when the subject has a bowel movement. See, e.g., Campieri M et al., Topical administration of 5-aminosalicylic acid enemas in patients with ulcerative colitis. Studies on rectal absorption and excretion, Gut, 1985 April; 26(4):400-5, the contents of which are incorporated herein by reference. Another issue with enemas is that the discomfort of providing and retaining the contents and the frequency with which they must be administered leads to low rates of patient compliance. Boyle, et al, Adherence to Rectal Mesalamine in Patients with Ulcerative Colitis, Inflamm Bowel Dis. 2015 December; 21(12):2873-8. doi. 10.1097/MIB.0000000000000562, the contents of which are incorporated herein by reference.

The invention provides methods that solve all of the aforementioned problems by using methods that allow delivery of high doses of therapeutic agents to the left colon. In addition, the methods afford superior retention of the agents in the colon. Consequently, the methods require much less frequent administration of enemas. As a result, the methods enable easier patient compliance, leading to better overall treatment of IBD.

Treating GI Conditions Using Multiple Dosing Regimens

Embodiments of the invention include methods of treating a GI condition using multiple, e.g., two or more, dosing regimens. The methods include providing an agent locally to the rectum or colon of a subject according to two or more dosing regimens that are different from each other. Additionally or alternatively, the methods may include providing an agent locally to the upper GI tract, e.g., mouth, esophagus, or stomach, of a subject according to two or more dosing regimens that are different from each other.

A dosing regimen may include one or more of a dosage, frequency of administration, mode of administration, and duration. The two dosing regimen may differ in dosage, frequency of administration, mode of administration, or any combination thereof.

The frequency of administration may be defined by the interval between doses. For example and without limitation, the interval between doses may be about 6 hours, about 8 hours, about 12 hours, about 15 hours, about 24 hours, about 36 hours, about 48 hours, about 60 hours, about 72 hours, about 84 hours, about 96 hours, about 5 days, about 6 days, about 7 days, about 8 days, about 10 days, about 12 days, about 14 days, about 3 weeks, about 4 weeks, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 10 days, at least 12 days, at least 14 days, at least 3 weeks, at least 4 weeks, greater than 6 hours, greater than 8 hours, greater than 12 hours, greater than 15 hours, greater than 24 hours, greater than 36 hours, greater than 48 hours, greater than 60 hours, greater than 72 hours, greater than 84 hours, greater than 96 hours, greater than 5 days, greater than 6 days, greater than 7 days, greater than 8 days, greater than 10 days, greater than 12 days, greater than 14 days, greater than 3 weeks, or greater than 4 weeks.

A dosing regimen may include one or more modes of administration. The mode of administration may be suitable for local administration or for systemic administration. For example and without limitation, modes of local administration include topical administration and rectal administration, e.g., via enemas, suppositories, foams, and other methods of delivery via the anus. For example and without limitation, modes of systemic administration include oral, enteral, parenteral, by injection, and by infusion.

In methods involving multiple phases, e.g., two or more phases, of treatment, the phases may differ by any relevant parameter. For example and without limitation, the phases may differ in one or more of duration, dosage, frequency of dose administration, mode of administration, route of administration, or therapeutic composition. The first phase of treatment may have a longer or shorter duration than the second phase of treatment. The first phase of treatment may have a higher or lower dosage of an agent than the second phase of treatment. The first phase of treatment may have a smaller or larger interval between dose administrations than the second phase of treatment. The first and second phases of treatment have the same mode of administration, or they may have different modes of administration. The first and second phases of treatment have the same route of administration, or they may have different routes of administration. The first and second phases of treatment employ the same composition, or they may employ different compositions. One phase of treatment may employ a single therapeutic agent, and another phase may employ a combination of therapeutic agents. The first and second phases of treatment may employ different combinations of therapeutic agents.

The subject may be an animal, such as a mammal. The subject may be a human, mouse, or rat.

The different dosing regimens may achieve distinct therapeutic goals. For example, the first dosing regimen may induce remission of the condition, and the second dosing may maintain remission of the condition.

Induction therapy is typically used to treat an acute phase of the condition or provide relief from symptoms associated with an acute phase. An acute phase of a condition may have one or more of the following features: abrupt onset, short duration, rapid progression, the need for urgent care, elevated levels of diagnostic markers. Acute phases of certain conditions, such as IBD, are known as “flare-ups”.

Maintenance therapy, on the other hand, generally prevents the condition or its symptoms from recurring. Maintenance therapy may be used for treatment of any non-acute phase of a condition, i.e., any phase of a condition that does not meet one or more criteria of an acute phase. Thus, maintenance therapy is usually long-term and continues even when the patient does not experience symptoms.

Methods of the invention may include providing an agent locally as described herein without the use of another form of therapy. Alternatively, methods of the invention may include providing an agent locally as described herein in combination with another form of therapy. For example and without limitation, the second form of therapy may differ in the agent, dosing regimen, dosage, dosing interval, mode of administration, route of administration, or any combination of the aforementioned elements. For example, the second form of therapy may include administration of an agent non-locally, e.g., systemically or orally. The second form of therapy may be performed prior to, concurrently with, or subsequent to providing an agent locally as described herein. Each therapeutic method may independently induce remission of the condition, maintain remission of the condition, or both.

Treating GI Conditions Following an Acute Phase

Embodiments of the invention include methods of treating a GI condition by providing an agent locally to the rectum or colon of a subject following an acute phase or flare of the condition. The treatment may maintain the GI condition in a reduced state. For example, the treatment may maintain remission of the condition. Additionally or alternatively, the methods may include treating a GI condition by providing an agent locally to the upper GI tract, e.g., mouth, esophagus, or stomach, of a subject following an acute phase or flare of the condition.

As indicated above, an acute phase or flare of a condition may have one or more of the following features: abrupt onset, short duration, rapid progression, the need for urgent care, elevated levels of diagnostic markers. Thus, treatments following an acute phase may entail treatment of any post-acute phase of a condition that does not meet one or more criteria of an acute phase.

The methods may include administering an agent locally according to a dosing regimen. The dosing regimen may include any of the elements described above in relation to dosing regimens, such as a dosage, frequency of administration, mode of administration, and duration.

As indicated above, the frequency of administration may be defined by the interval between doses. For example and without limitation, the interval between doses may be about 6 hours, about 8 hours, about 12 hours, about 15 hours, about 24 hours, about 36 hours, about 48 hours, about 60 hours, about 72 hours, about 84 hours, about 96 hours, about 5 days, about 6 days, about 7 days, about 8 days, about 10 days, about 12 days, about 14 days, about 3 weeks, about 4 weeks, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, at least 8 days, at least 10 days, at least 12 days, at least 14 days, at least 3 weeks, at least 4 weeks, greater than 6 hours, greater than 8 hours, greater than 12 hours, greater than 15 hours, greater than 24 hours, greater than 36 hours, greater than 48 hours, greater than 60 hours, greater than 72 hours, greater than 84 hours, greater than 96 hours, greater than 5 days, greater than 6 days, greater than 7 days, greater than 8 days, greater than 10 days, greater than 12 days, greater than 14 days, greater than 3 weeks, or greater than 4 weeks.

As described above, a dosing regimen may include one or more modes of administration. The mode of administration may be suitable for local administration or for systemic administration. For example and without limitation, modes of local administration include topical administration and rectal administration, e.g., via enemas, suppositories, foams, and other methods of delivery via the anus. For example and without limitation, modes of systemic administration include oral, enteral, parenteral, by injection, and by infusion.

The subject may be any type of subject, as described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Treating GI Conditions Using Extended Intervals Between Doses

Embodiments of the invention include treating a GI condition by repeatedly providing an agent locally to the rectum or colon of a subject in which the doses are separated by extended intervals. Additionally or alternatively, the methods may include treating a GI condition by repeatedly providing an agent locally to the upper GI tract, e.g., mouth, esophagus, or stomach, of a subject in which the doses are separated by extended intervals.

A problem with prior methods of topical administration of therapeutic agents to the colon is that the poor retention of the agent in the colon following bowel movements necessitates frequent re-administration of the agent via enema, the inconvenience of which leads to low rates of patient compliance with prescribed dosing regimens. See, e.g., Boyle, et al, Adherence to Rectal Mesalamine in Patients with Ulcerative Colitis, Inflamm Bowel Dis. 2015 December; 21(12):2873-8. doi: 10.1097/M1B.0000000000000562, the contents of which are incorporated herein by reference. Due to the superior retention of therapeutic agents in the colon using the compositions and methods of the invention, embodiments of the invention allow subjects extended intervals between administrations of enemas, leading to better patient compliance.

The interval between doses may be a defined period. For example and without limitation, the interval between doses may be about 6 hours, about 8 hours, about 12 hours, about 24 hours, about 36 hours, about 48 hours, about 60 hours, about 72 hours, about 84 hours, about 96 hours, about 5 days, about 6 days, about 7 days, at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The agent may be retained in the colon for a defined period. For example and without limitation, the agent may be retained in the colon for at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The agent may exert or maintain a therapeutic effect in the colon for a defined period. For example and without limitation, the agent may exert or maintain a therapeutic effect in the colon for at least 6 hours, at least 8 hours, at least 12 hours, at least 15 hours, at least 24 hours, at least 36 hours, at least 48 hours, at least 60 hours, at least 72 hours, at least 84 hours, at least 96 hours, at least 5 days, at least 6 days, at least 7 days, greater than 6 hours, greater than 8 hours, greater than 12 hours, greater than 24 hours, greater than 36 hours, greater than 48 hours, greater than 60 hours, greater than 72 hours, greater than 84 hours, greater than 96 hours, greater than 5 days, greater than 6 days, or greater than 7 days.

The methods may include administering an agent locally according to a dosing regimen. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Treating GI Conditions without Repeating Doses Following Bowel Movements

Embodiments of the invention include methods in which an agent is provided locally to the rectum or colon of a subject, and the agent is not re-administered to the subject following a bowel movement but maintains its therapeutic effect following the bowel movement. Clearance from the colon is a problem with prior methods of administration of therapeutic agents. For example, when 5-ASA is orally administered to subjects, levels of 5-ASA in the colon are decreased by laxative or colonic lavage. De Vos, et al., Concentrations of 5-ASA and Ac-5-ASA in human ileocolonic biopsy homogenates after oral 5-ASA preparations, Gut, 1992 October; 33(10):1338-42, the contents of which are incorporated herein by reference. When 5-ASA is applied topically by enema, colonic 5-ASA decreases dramatically following a bowel movement. Campieri et al., Topical administration of 5-aminosalicylic acid enemas in patients with ulcerative colitis, Studies on rectal absorption and excretion, Gut, 1985, 26, 400-405, the contents of which are incorporated herein by reference.

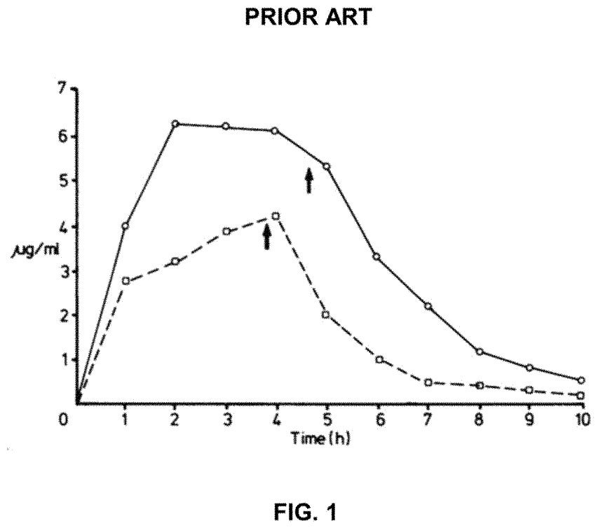

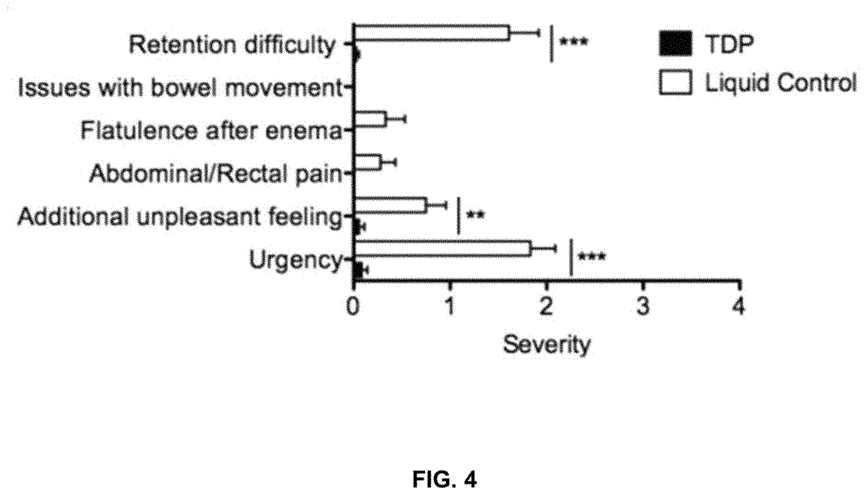

FIG. 1 is graph showing plasma levels of 5-ASA in two patients following administration by enema according to a prior art method. Campieri, et al., FIG. 4. Arrows indicate time points when patients evacuated their bowels. The results show that bowel movements dramatically lower systemic levels of 5-ASA when the drug is administered by enema according to prior methods.

Methods and compositions of the invention provide stable delivery of therapeutic agents that are retained in the colon even after evacuation of the bowels. Therefore, the invention provides methods in which topical administration of a therapeutic agent does not need to be repeated after the subject moves his bowels.

For example, in certain methods of the invention, efficacy or drug absorption is not hindered or is minimally hindered, i.e., not hindered beyond a defined threshold, after the subject has a bowel movement. In certain methods of the invention, efficacy, drug absorption, and/or drug levels are maintained above a defined threshold after the subject has a bowel movement. Consequently, compared to prior methods, methods of the invention are less burdensome.

A defined amount of the agent may be retained in the colon following a bowel movement. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the agent may be retained in the colon following a bowel movement by the subject.

A defined therapeutic effect of the agent may be maintained in the colon following a bowel movement. For example, at least 10%, at least 20%, at least 30%, at least 40⁰/%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the therapeutic effect may be maintained in the colon following a bowel movement by the subject.

In methods that do not require re-administration of an agent following a bowel movement, the agent may nonetheless be re-administered at an interval that is independently defined and not dependent on the subject's bowel movements. For example, the agent may be re-administered after one of the intervals described above or according to a dosing regimen described above. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Treating GI Conditions without Repeating Doses Following Consumption of Food or Liquid

As described below, methods of the invention are also useful for treating conditions of the upper GI tract, such as eosinophilic esophagitis, oral mucositis, and esophageal varices. In certain embodiments, the invention provides methods and compositions that are retained in the upper GI tract, e.g., mouth, esophagus, or stomach, even after consumption of liquids or solid food. Therefore, the invention provides methods in which topical administration of a therapeutic agent does not need to be repeated after the subject eats and/or drinks.

A defined amount of the agent may be retained in the upper GI tract following consumption of food, liquid, or both. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the agent may be retained in the upper GI tract following consumption of food, liquid, or both.

A defined therapeutic effect of the agent may be maintained in the upper GI tract following consumption of food, liquid, or both. For example, at least 10%, at least 20%, at least 30%, at least 40%, at least 50%, at least 60%, at least 70%, at least 80%, at least 90%, greater than 10%, greater than 20%, greater than 30%, greater than 40%, greater than 50%, greater than 60%, greater than 70%, greater than 80%, or greater than 90% of the therapeutic effect may be maintained in the upper GI tract following consumption of food, liquid, or both.

In methods that do not require re-administration of an agent following consumption of food, liquid, or both, the agent may nonetheless be re-administered at an interval that is independently defined and not dependent on eating or drinking by the subject. For example, the agent may be re-administered after one of the intervals described above or according to a dosing regimen described above. The dosing regimen may include any of the elements described above in relation to dosing regimens, such as a dosage, frequency of administration, mode of administration, and duration.

The subject may be any type of subject described above. The subject may be a human.

The methods may include providing an agent locally as described herein without the use of another form of therapy. Alternatively, methods of the invention may include providing an agent locally as described herein in combination with another form of therapy. The second form of therapy may have any of the elements described above.

Compositions for Use in Treating GI Conditions

Methods of the invention include providing an agent locally to the rectum or colon of a subject. The agent may be effective for treating inflammation or infection associated with IBD.

For example and without limitation, the agent may be an aminosalicylate, angiotensin receptor inhibitor, anti-inflammatory, antibiotic, antibody, antimetabolite, antimycotic, antisense oligonucleotide, bacterial sample, beta-blocker, biologic, budesonide, calcineurin inhibitor, corticosteroid, cytokine, growth factor, immunosuppressor, indole, interferon, Janus kinase inhibitor, microbial metabolite, mTOR inhibitor, opioid, PDE4 inhibitor, peptide, pregnane X receptor (PXR) ligand, probiotic, protein, proton pump inhibitor (PPI), salicylic acid derivative, small molecule, sphingosine-1-phosphate receptor modulator, thiopurine, TNF-α binding protein, or toll-like receptor (TLR) ligand.

The aminosalicylate may be 5-ASA, 4-ASA, azodisalicylate, balsalazide, ipsalazide, olsalazine, or sulfasalazine.

The angiotensin receptor inhibitor may be candesartan, eprosartan, fimasartan, irbesartan, losartan, olmesartan, saprisartan, telmisartan, orvalsartan.

The antimycotic may be abafungin, albaconazole, amorolfin, amphotericin B, anidulafungin, bifonazole, butenafine, butoconazole, candicidin, caspofungin, clotrimazole, econazole, efinaconazole, epoxiconazole, fenticonazole, filipin, fluconazole, hamycin, isavuconazole, isoconazole, itraconazole, ketoconazole, luliconazole, micafungin, miconazole, naftifine, natamycin, nystatin, omoconazole, oxiconazole, posaconazole, propiconazole, ravuconazole, rimocidin, sertaconazole, sulconazole, terbinafine, terconazole, tioconazole, or voriconazole.

The beta blocker may be acebutolol, atenolol, betaxolol, bisoprolol, bucindolol, butaxamine, carteolol, carvedilol, celiprolol, esmolol, ICI-118,551, labetalol, metoprolol, nadolol, nebivolol, oxprenolol, penbutolol, pindolol, propanolol, sotalol, SR 59230A, or timolol.

The biologic may be a TNF-alpha inhibitor, such as adalimumab, certolizumab pegol, golimumab, or infliximab; an integrin receptor antagonist, such as natalizumab or vedolizumab; or an interleukin antagonist, such as ustekinumab. The biologic may be a biosimilar of another biologic.

The calcineurin inhibitor may be ciclosporin, pimecrolimus, or tacrolimus.

The corticosteroid may be beclamethasone, budesonide, dexamethasone, prednisolone, or prednisone.

The immunosuppressor may be 6-mercaptopurine, 6-thioguanine, azathioprine, cyclosporine, methotrexate, mycophenolate, or tacrolimus.

The indole may be indole, indole 3 acetic acid (IAA), or indole 3 propionic acid (IPA).

The interferon may be interferon alfa-2a, interferon alfa-2b, interferon alfacon-1, interferon alfa-n3, interferon beta-1a, interferon beta-1b, interferon gamma-1b, peginterferon alfa-2a, peginterferon alfa-2b, peginterferon alfa-2b, or peginterferon beta-1a.

The Janus kinase (JAK) inhibitor may be filgotinib, peficitinib, tofacitinib, upadicitinib, Pf-06651600, Pf-06700841, or TD-1473.

The microbial metabolite may be indole, indole 3 acetic acid (IAA), or indole 3 propionic acid (IPA).

The mTOR inhibitor may be everolimus, sirolimus, or temsirolimus.

The opioid may be alfentanil, carfentanil, etorphine, fentanyl, hydromorphone, morphine, pethidine, remifentanil, or sufentanil.

The PDE4 inhibitor may be apremilast, cilomilast, crisaborole diazepam, ibudilast, luteolin, mesembrenone, piclamilast, roflumilast, or rolipram.

The pregnane X receptor (PXR) ligand may be BAS451, FKK1, FKK2, FKK3, FKK4, FKK5, FKK6, FKK7, FKK8, FKK9, FKK10, FKK999, hyperforin, rifampicin, rifaximin, or SR12813.

The proton pump inhibitor (PPI) may be dexlansoprazole, esomeprazole, lansoprazole, omeprazole, pantoprazole, or rabeprazole.

The salicylic acid derivative may be 5-aminosalicylic acid, or a derivate/variant, such as 4-aminosalicylic acid, azodisalicylate, balsalazide, ipsalazide, olsalazine, or sulfasalazine.

The sphingosine-1-phosphate receptor modulator may be fingolimod, laquinimod, ozanimod, ponesimod, or siponimod.

The thiopurine may be 6-mercaptopurine (6-MP), 6-thioguanine (6-TG), or azathioprine (AZA).

The TNF-α binding protein may be adalimumab, certolizumab pegol, etanercept, golimumab, or infliximab.

The TLR ligand may be 3-deoxy-D-manno-octulosonic acid (KDO2)-lipid A.

The agent may be provided in a formulation that exists as a liquid when the formulation is below a threshold condition and as a gel when the formulation is above threshold condition.

The threshold may be any combination of physical, chemical, and temporal conditions.

The chemical condition may be acidity, alkalinity, or pH. The threshold condition may be a transition pH. The formulation may exist as a liquid when the formulation is below the transition pH and as a gel when the formulation is above the transition pH. The threshold condition may be a transition pH. The formulation may exist as a liquid when the formulation is above the transition pH and as a gel when the formulation is below the transition pH.

The temporal condition may be time. The threshold condition may be a transition time point. The formulation may exist as a liquid prior to the transition time point and as a gel after the transition time point.

The physical condition may be temperature. The threshold condition may be a transition temperature. The formulation may exist as a liquid when the formulation is below the transition temperature and as a gel when the formulation is above the transition temperature. The formulation may exist as a liquid when the formulation is above the transition temperature and as a gel when the formulation is below the transition temperature.

The agent may be provided in a formulation that exists as a liquid at a first temperature and transitions to a gel at a second temperature. The transition from the first temperature to the second temperature may be accompanied by an increase in viscosity. For example, the formulation may exist as a liquid at or near room temperature (about 23° C.) and as a gel at or near physiological temperature (about 37° C.). When such formulations are stored and administered to a patient at room temperature, e.g., via enema, they can readily reach the rectum, sigmoid colon, and descending colon. Once inside the colon, such formulations transition to a gel phase and adhere to the lining of the colon, thereby allowing prolonged exposure of the inflamed tissue to the agent. Formulations that make such phase transitions and their use for delivery of therapeutic agents to the colon are described in, for example, International Patent Publication No. WO 2016/179227; and Sidhartha R. Sinha, et al., A Thermo-Sensitive Delivery Platform for Topical Administration of Inflammatory Bowel Disease Therapies, Gastroenterology, 2015 July; 149(1):52-55.e2, doi: 10.1053/j.gastro.2015.04.002, the contents of each of which are incorporated herein by reference.

For example and without limitation, the formulation may exist as a liquid at about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., or about 35° C.

For example and without limitation, the formulation may exist as a liquid at about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

For example and without limitation, the formulation may transition to a gel at from about 16° C. to about 40° C., from about 18° C. to about 40° C., from about 20° C. to about 40° C., from about 22° C. to about 40° C., from about 24° C. to about 40° C., from about 26° C. to about 40° C., from about 28° C. to about 40° C., from about 30° C. to about 40° C., from about 32° C. to about 40° C., from about 34° C. to about 40° C., from about 36° C. to about 40° C., from about 38° C. to about 40° C., from about 16° C. to about 38° C., from about 18° C. to about 38° C., from about 20° C. to about 38° C., from about 22° C. to about 38° C., from about 24° C. to about 38° C., from about 26° C. to about 38° C., from about 28° C. to about 38° C., from about 30° C. to about 38° C., from about 32° C. to about 38° C., from about 34° C. to about 38° C., from about 36° C. to about 38° C., from about 16° C. to about 36° C., from about 18° C. to about 36° C., from about 20° C. to about 36° C., from about 22° C. to about 36° C., from about 24° C. to about 36° C., from about 26° C. to about 36° C., from about 28° C. to about 36° C., from about 30° C. to about 36° C., from about 32° C. to about 36° C., from about 34° C. to about 36° C., from about 16° C. to about 34° C., from about 18° C. to about 34° C., from about 20° C. to about 34° C., from about 22° C. to about 34° C., from about 24° C. to about 34° C., from about 26° C. to about 34° C., from about 28° C. to about 34° C., from about 30° C. to about 34° C., from about 32° C. to about 34° C., from about 16° C. to about 32° C., from about 18° C. to about 32° C., from about 20° C. to about 32° C., from about 22° C. to about 32° C., from about 24° C. to about 32° C., from about 26° C. to about 32° C., from about 28° C. to about 32° C., from about 30° C. to about 32° C., from about 16° C. to about 30° C., from about 18° C. to about 30° C., from about 20° C. to about 30° C., from about 22° C. to about 30° C., from about 24° C. to about 30° C., from about 26° C. to about 30° C., from about 28° C. to about 30° C., from about 16° C. to about 28° C., from about 18° C. to about 28° C., from about 20° C. to about 28° C., from about 22° C. to about 28° C., from about 24° C. to about 28° C., from about 26° C. to about 28° C., from about 16° C. to about 26° C., from about 18° C. to about 26° C., from about 20° C. to about 26° C., from about 22° C. to about 26° C., from about 24° C. to about 26° C., from about 16° C. to about 24° C., from about 18° C. to about 24° C., from about 20° C. to about 24° C., from about 22° C. to about 24° C., from about 16° C. to about 22° C., from about 18° C. to about 22° C., from about 20° C. to about 22° C., from about 16° C. to about 20° C., or from about 18° C. to about 20° C.

The agent may be provided in a formulation that transitions between a liquid phase and a gel phase in response to a stimulus other than, or in addition to, a change in temperature. For example and without limitation, the stimulus may be or include one or more of a change in pH, solvent exchange, electromagnetic radiation (e.g., visible light, ultraviolet, infrared, X-rays, fluorescence), sound (e.g., ultrasound), pressure, or the presence of specific ions or molecules. Examples of systems that exhibit in situ sol-gel transitions are known in the art and described in, for example, Kouchak, M., In Situ Gelling Systems for Drug Delivery, Jundishapur J Nat Pharm Prod. 2014 August; 9(3): e20126, PMCID: PMC4165193; PMID: 25237648; and Jones and Steed, Gels with sense: supramolecular materials that respond to heat, light and sound, Chem. Soc. Rev., 2016, 45, 6546-6596, DOI 10.1039/C6CS00435K, the contents of each of which are incorporated herein by reference.

The formulation may contain an agent in mixture with a polymer. The agent may be in a complex with, or conjugated to, the polymer. Without wishing to be bound by theory, the polymer may form micelles that retain the agent in the formulation in the liquid phase. At higher temperatures, dehydration of the micelles may cause them to aggregate to form a gel while releasing the agent so that it can enter the tissue of the colon.

The polymer may be a block copolymer. The block polymer may include blocks of a relatively hydrophilic polymer, such as polyethylene glycol, and blocks of a relatively hydrophobic polymer, such as polypropylene glycol.

The polymer may be a natural polymer. For example and without limitation, the natural polymer may be pectin, xyloglucan, gellan gum, chitosan, or alginic acid.

The polymer may be an inorganic polymer. For example and without limitation, the polymer may be or contain silicon oxide.

The formulation may contain a lipid. The lipid may be a fatty acid, glycolipid, phosphoglyceride, phospholipid, sphingolipid, or sterol. For example and without limitation, the lipid may be glucosyl-cerebroside, phosphatidylcholine, phosphatidylethanolamine, phosphatidylinositol, phosphatidylserine, sphingomyelin.

The formulation may contain one or more molecules that exist as free molecules in solution but form polymers in response to a stimulus, thereby promoting transition to a gel phase. Such molecules may be organic molecules, inorganic molecules, or macromolecule, e.g., proteins. For example, under acidic conditions certain mucins undergo a conformational change that allows them to polymerize. Consequently, reducing the pH of concentrated mucin solutions can trigger gel formation. In another example, mixtures of polyacrylic acid and guanidium exist as liquid solutions at pH of ≤4 but transition to gels at neutral pH due to the formation of a supramolecular poly-electrolyte complex (SPEC). For example and without limitation, the stimulus that promotes polymerization and/or gel formation may be a divalent cation, e.g., calcium or magnesium, an epoxy, an acid, or a base.

GI Conditions

Methods of the invention may be used to treat any GI condition. For example and without limitation, the GI condition may be achalasia, Barrett's esophagus, Boerhaave syndrome, celiac disease, constipation, Crohn's disease, diverticulitis, enteritis, enterocolitis, eosinophilic esophagitis, esophageal burns, esophageal candidiasis, esophageal spasm, esophageal stricture, esophageal webbing, esophageal varices, esophagitis, gastritis, gastritis, gastroenteritis, gastroesophageal reflux disease, gastrointestinal bleeding, indeterminate colitis, inflammatory bowel disease, intestinal graft-versus-host disease, irritable bowel syndrome, Mallory-Weiss tears, microscopic colitis, nutcracker esophagus, oral mucositis, pernicious anemia, pouchitis, radiation colitis, radiation esophagitis, radiation proctitis, ulcerative colitis, ulcers, or Zenker's diverticulum.

Certain methods of the invention are useful for treatment of IBD. IBD is a group of debilitation conditions, including Crohn's disease, ulcerative colitis, and indeterminate colitis. IBD occurs when tissue in the GI tract becomes inflamed. Crohn's disease may affect tissue of the mouth, esophagus, stomach, small intestine, large intestine, or anus. Ulcerative colitis primarily affects the colon and the rectum. Inflammation may be localized or concentrated in one or more specific parts of the colon, such as the ascending colon, transverse colon, descending colon, sigmoid colon, or the rectum. Indeterminate colitis includes colitis that is deemed not to be either Crohn's disease or ulcerative colitis and may have some features of either or both. IBD may be accompanied by one or more symptoms, such as abdominal pain, anemia arthritis, bronchiolitis obliterans organizing pneumonia, cramps/muscle spasms, deep vein thrombosis (DVT), diarrhea, fatigue, fever, loss of appetite, non-thyroidal illness syndrome (NTIS), primary sclerosing cholangitis, pyoderma gangrenosum, erythema nodosum, arthritis, and rectal bleeding.

Treatment of IBD typically involves two phases. In the first phase (induction), the goal of treatment is to induce remission of the inflammation and provide relief from symptoms. Induction treatment is used during an acute phase or flare of IBD. Once remission has been achieved, the second phase (maintenance) of treatment is directed toward maintaining remission and preventing relapse. Maintenance therapy is generally long-term and continues even when the patient does not experience symptoms. Thus, maintenance therapy is used to maintain the IBD in a reduced state. Induction and maintenance phases may involve the same or different medications, modes of administration, frequencies of administration, and dosages.

An acute phase of IBD may have one or more of the following features described above in relation to acute phases of conditions generally. As indicated above, acute phases of IBD are sometimes called as “flare-ups”. The following markers may be used to diagnose IBD, determine its level of severity, and characterize the phase of the condition, e.g., determine whether it is acute or non-acute: albumin, anti-neutrophil cytoplasmic antibody (ANCA), anti-Saccharomyces cerevisiae antibodies (ASCA), C reactive protein (CRP), calprotectin, erythrocyte sedimentation rate (ESR), lactoferrin, leucocyte count, platelet count, and α1 acid glycoprotein (orosomucoid). IBD can also be evaluated by analysis of proteome, transcriptome, genome, and post-translational modifications, such as phosphorylation, acetylation, glycosylation, disulfide bond formation, deamidation, and citrullination. Biomarkers of IBD and their diagnostic application are described in more detail in, for example, Bennike T. et al., Biomarkers in inflammatory bowel diseases: Current status and proteomics identification strategies, World J Gastroenterol. 2014 Mar. 28; 20(12): 3231-3244, doi: 10.3748/wjg.v20.i12.3231; Mohsen Norouzinia et al., Biomarkers in inflammatory bowel diseases: insight into diagnosis, prognosis and treatment, Gastroenterol Hepatol Bed Bench, 2017 Summer; 10(3): 155-167; and Viennois E., et al., Biomarkers of IBD: from classical laboratory tools to personalized medicine, Inflamm Bowel Dis. 2015 October; 21(10): 2467-2474, doi: 10.1097/MIB.0000000000000444, the contents of each of which are incorporated herein by reference.

Methods of the invention are also useful for treatment of irritable bowel syndrome (IBS). IBS comprises GI symptoms, such as abdominal pain and changes in the pattern of bowel movements, without any evidence of underlying damage. Symptoms may occur over a period of years. IBS has been classified into the following four types based on whether diarrhea and constipation are common: IBS-D, in which diarrhea is common; IBS-C, in which constipation is common; IBS-M, in which both diarrhea and constipation are common; and IBS-U, in which neither diarrhea nor constipation is common.

Methods of the invention may also be used to treat conditions of the upper GI tract. For example and without limitation, methods may include treatment of eosinophilic esophagitis, oral mucositis, or esophageal varices.

### EXAMPLES

The following examples help to describe embodiments of the invention. The examples are provided for illustrative purposes only and are not intended to limit the scope of the invention.

### Example 1

The retention and duration of therapeutic effect of agents delivered topically to the colon were tested in mouse model of IBD. Colitis was induced in mice by addition of 2% dextran sulfate to their drinking water for 9 days. On day 6, when bloody stools were apparent, 150 μL of BL/barium (5% barium sulfate suspension in a 0.1 mg/mL budesonide solution) or BPL/barium (5% barium sulfate suspension in a homogeneous solution containing 0.4 mg/mL 1,2-distearoyl-sn-glycero-3-phosphocholine and 0.1 mg/mL budesonide) was administered by enema. Mice were allowed to defecate normally following enema administration. Tomographic images were taken 0.5 hours, 1.5 hours, and 3.0 hours after enema administration.

Contents delivered by enema showed improved residency time in the colon in mice given BPL compared to mice given BL. BPL-treated mice had better drug absorption, and contents were retained in the colon even following bowel movements. BPL-treated mice also showed improved mucosal healing compared to BL-treated mice, with 50× more drug reaching tissue and faster treatment of inflammation.

The results show that enematic delivery of compositions containing a therapeutic agent in a homogeneous polymeric composition allows retention of the agent in the colon following bowel movements and that the retained agent continues to exert a therapeutic effect to ameliorate inflamed mucosal tissue. The results further show that such methods are effective at treating a mouse model of colitis and suggest that they may be useful for treating IBD in humans as well.

### Example 2

### Introduction

Topical therapy to the gastrointestinal tract is highly effective for the administration of therapies for millions. Yet, these rectally administered treatments remain underutilized in a diverse spectrum of patients from children and adults suffering from inflammatory bowel disease (IBD) to those undergoing treatment where oral or parenteral administration may not be possible, desired, or cost-effective, such as hospice. While topical therapy in its current form has drawbacks, including issues with convenience, frequency, retention, and urgency, it also offers many advantages over other means of therapy. In children and adults suffering from IBD, for example, topical therapy allows for direct administration of therapy to the site of disease and has been shown to be highly effective in the treatment of IBD, either on its own or as adjunctive therapy. Oral or parenteral systemic agents are also effective and often used to treat IBD, but many of these immunosuppressive agents have very serious side effects, including increased risk of multiple malignancies and serious infections.

While topical therapy can be used for many diseases, it is most established for use in patients suffering from gastrointestinal disorders such as IBD. In the US alone, 3 million people have IBD, either Crohn's disease (CD) or ulcerative colitis (UC), and millions more suffer globally with increasing prevalence. Both diseases are lifelong, relapsing illnesses that typically strike young patients and require induction followed by maintenance therapy. These patients often have many debilitating symptoms and are at increased risk of colorectal cancer, particularly with uncontrolled disease. Up to 70% of patients with UC and a smaller number of patients with CD have disease limited to the left-side of the colon or distal colitis. Patients with this distribution of disease are particularly suitable for topical therapy, as therapeutics can be delivered rectally to the entire distal colon. Also, topical therapy is often used in conjunction with systemic therapies such as oral or injectable treatments. While systemically delivered agents can provide treatment for proximal areas of disease beyond the reach of topical therapy, disease in UC is frequently most severe distally and may require additional topical therapy to obtain complete remission, particularly during disease flares.

The mainstay of topical therapy is the enema. Despite its advantages, patients with distal colitis (those who would benefit most) are often unable to tolerate enemas due to urgency/spasm and the associated inability to retain a liquid solution for the recommended dosing and time frame of several hours, once or twice a day. Other topicals include foam or suppository preparations. These options tend to be easier to retain but have the marked disadvantage of being unable to reach proximal areas of the left colon that are accessible with an enema. See, e.g., Brunner, et al., Colonic spread and serum pharmacokinetics of budesonide foam in patients with mildly to moderately active ulcerative colitis, Aliment Pharmacol Ther 2005; 22: 463-470, doi: 10.1111/j.1365-2036.2005.02571.x, the contents of which are incorporated herein by reference. These limitations of existing therapy and inconvenience cause many patients to not benefit from topical therapy's potential.

To precisely address these issues, we have developed a thermosensitive delivery platform (TDP) using Food and Drug Administration generally recognized as safe (GRAS) components. TDP can be self-administered at room temperature as a liquid solution, thereby maintaining the proximal delivery advantage of liquid enemas. At body temperature in vivo the formulation quickly transitions to a viscous gel, similar to the consistency of toothpaste, without expanding in volume. As such, TDP is designed to overcome issues with tolerability known to be formidable concerns of enema users. The viscous gel enhances the ability to retain the formulation and is designed to improve ease of use and acceptance by patients with IBD. The mucoadhesive and temperature-dependent gelation properties allow the formulation to coat the colon wall, optimizing drug delivery and exposure time of the therapeutic being delivered with TDP. During bowel movements, the natural peristaltic movement of the colon evacuates the gel.

We have tested TDP in the delivery of a variety of therapeutics in experimental colitis and have shown TDP to be more effective than conventionally delivered therapeutics and appropriate controls. In addition, TDP can be retained longer than standard liquid enema therapy in animal models, allowing more time for drug to be delivered to inflamed mucosa and suggesting some mucoadhesive abilities. In this study, we conducted first-in-human studies of TDP alone (without an active therapeutic) to test our hypothesis that TDP will be preferred by subjects over control enema (water). In addition, we posit that TDP would achieve as good proximal distribution in the colon as liquid control. To test these hypotheses, we designed a randomized double-blind, placebo-controlled crossover study where healthy subjects were given both TDP and liquid control (with contrast) on different days by a gastrointestinal radiologist. Following the enema administration, the subjects reported their preference and side effects in a user questionnaire. Abdominal x-rays were performed to assess proximal distribution of both enemas and the maximum proximal distance reached was determined by a gastrointestinal radiologist. The radiologist delivering the enemas as well as evaluating the proximal distance radiographically was blinded to the treatment assignment and formulation type, both liquid enemas at room temperature.

Methods: Study Design

This study was a prospective, randomized, double-blind, placebo controlled, crossover study. The study was registered on clinicaltrials.gov (NCT02290665) and approved by the Stanford University Institutional Review Board (29620). Written informed consent was obtained from all participants.

Methods: Study Participants

Subjects were recruited from Stanford University between January and October 2015. Broadly, inclusion criteria were healthy male and non-pregnant female volunteers between 18 and 70 years of age. Subjects with gastrointestinal complaints such as rectal bleeding, tenesmus, urgency, fecal incontinence, or with active or prior history of IBD were excluded from this study.

Methods: Study Intervention

Subjects were randomized into two groups without restrictions using a Microsoft Excel-generated randomization list. The randomization list was generated by a single co-investigator prior to enrolling any subjects. Subjects were then enrolled by a study coordinator and enemas were delivered by a blinded gastrointestinal radiologist. All subjects enrolled completed the study. Group 1 received TDP formulation first, followed by the water enema. Group 2 received the control enema first, followed by the TDP formulation. The two formulations were given on separate days with at least 3 days in between enemas. Subjects and all investigators were masked to the order of the formulations.

In the first study visit, a gastrointestinal radiologist administered the first formulation (TDP formulation if in group 1, control enema if in group 2). A plain abdominal x-ray was taken immediately after instillation to assess proximal distribution. While retaining the formulation, subjects were asked to fill out a questionnaire to evaluate a variety of parameters related to the tolerability, side effects, and ease of retention of the formulation. Subjects were asked to try to retain the enema for at least one hour.

Subjects returned for the second study at least 3 days after the initial visit for the second enema. Similar to the initial visit, in the second study visit, the subjects were given the other enema by a gastrointestinal radiologist blinded to the type of enema. For the second enemas, the control enema was given to subjects in group 1 and TDP enema was given to subjects in group 2. Again, subjects were asked to fill out the questionnaire (including formulation preference) while retaining the formulation. Three subjects had a second x-ray after each enema to assess the distribution of the enema about 6 hours after time of instillation. All subjects were asked if they would consider the second x-ray after each enema. Only three subjects agreed to additional imaging. All subjects were recruited and completed testing between January and September 2015.

Methods: Formulations

Each subject was given both formulations at different time points as noted above. The TDP formulation consisted of 100 ml total volume: 20% (mass by volume) of polymer, 10 ml Cystografin contrast (Bracco Diagnositics), and deionized water sufficient to bring total volume to 100 ml (Koshland Pharmacy, San Francisco, Calif.). The control formulation consisted of 90 ml of tap water with 10 ml of Cystografin contrast. The minimum amount of contrast needed was determined with pre-clinical tests, in which various concentrations of contrast were added to the formulation and test x-rays were performed using an abdominal imaging phantom to determine the least amount of contrast needed to visualize the enema radiographically.

Methods: Imaging

To compare the depth of spread of the two formulations, subjects received both TDP and control formulations with contrast agent and underwent fluoroscopy-assisted plain film radiographic imaging immediately following the administration of the formulations. This allowed for evaluation of the proximal depth of spread of TDP vs. control enema. An additional x-ray was taken in a subset of three subjects approximately 6 hours after the first enema (for a maximum of 4 sets of radiographic images in these three subjects). X-rays were evaluated by a single gastrointestinal radiologist and evaluated for maximum proximal distance in a blinded fashion. The maximum extent of formulation or liquid control was noted anatomically (descending colon, sigmoid colon, recto-sigmoid colon, or rectum).

Methods: Endoscopy and Plasma Detection

Two subjects underwent flexible sigmoidoscopy within 2 hours of receiving TDP or liquid control enema in order to visually assess the colonic mucosa and obtain tissue biopsies. These subjects did not receive any bowel preparation. Biopsies were read by a pathologist blinded to the intervention.

Blood was drawn from the subject one hour after enema and the concentration of the polymer in plasma was determined by forming a water-insoluble polymer cobalt complex using cobalt(II) thiocyanate. The complex was re-dissolved in acetonitrile, and absorbance was measured at 624 nm using a UV-Vis plate reader (Molecular Devices—FlexStation® 3), which is proportional to the polymer-cobalt concentration and described in the literature.

Methods: Immunohistochemistry

Colon tissue biopsies were fixed in methanol-Carnoy's fixative (60% (v/v) dry methanol, 30% (v/v) chloroform, 10% (v/v) glacial acetic acid) for 6 hours and embedded in paraffin. Samples were then stained using mucin 2 rabbit polyclonal IgG primary antibody (Santa Cruz Biotechnology) (1:100) and Alexa Fluor 488 Donkey Anti rabbit IgG secondary antibody (Jackson ImmunoResearch Laboratories) (1:250) as described in the literature.

Methods: Study Outcomes

The primary outcome of this study was to investigate the preference of TDP compared to liquid control enema. In addition, we sought to determine if TDP would be delivered as proximally in the colon compared to liquid control and to compare side effects.

Methods: Statistical Analysis

A minimum sample size of 12 was deemed necessary to have our study appropriately powered, based on earlier results comparing the subject preference of foam over control enema. Using a likelihood ratio test for two independent proportions, a sample size of 12, assuming similar preference proportions of 84% TDP vs. 6% control enema and alpha of 5%, was determined to provide 80% power to reject the null hypothesis of no preference between the two enemas. 18 patients (50% more than minimum sample size) were enrolled to account for the possibility of reduced preference of TDP compared to foam. All authors had access to the study data and had reviewed and approved the final manuscript.

Results: TDP Reduces Adverse Effects and is Preferred Over Standard Liquid Enema

Improving topical delivery requires overcoming some of the key limitations of existing therapy. We have previously shown the superiority of TDP in delivering therapeutics topically in two animal colitis models. However, animal models for rectal delivery cannot, of course, predict preference. To assess this, we designed a randomized double-blind crossover study to determine subject preference and compare adverse effects of TDP versus a liquid control.

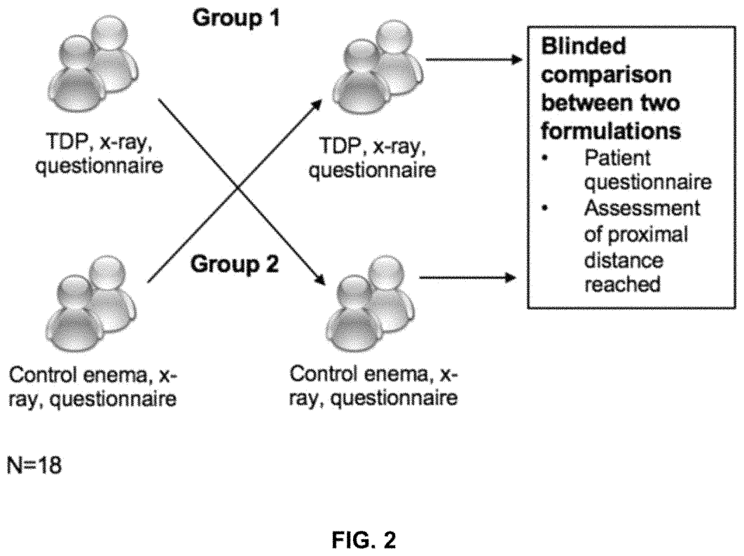

FIG. 2 is a schematic of the randomized double-blind, placebo-controlled crossover study design used to assess thermosensitive delivery platform (TDP) versus liquid control enema. Eighteen healthy adult subjects were enrolled in the study and randomized to start with either TDP or control enema (both with a small amount of iodinated contrast). Following administration of the enema (100 ml), an abdominal x-ray was performed. Subsequently, subjects were asked to complete a questionnaire documenting their preference and other characteristics regarding their experience after each enema. Several days after receiving the first enema (and obtaining abdominal x-ray and completing post-enema questionnaire), subjects had the process (enema, x-ray, questionnaire) repeated with the other enema. Both the radiologist administering the enema and the subject were blinded to the type of enema (liquid control or TDP) that was administered.

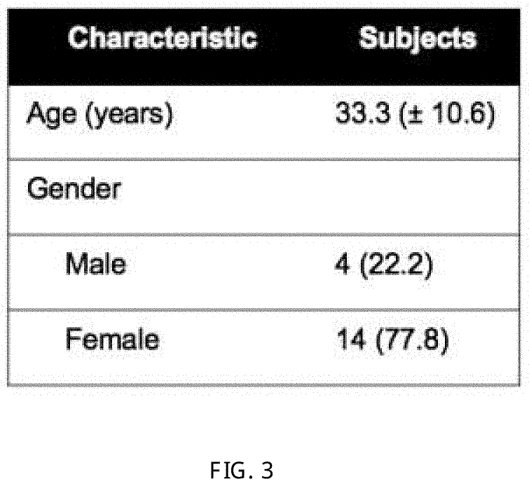

FIG. 3 is a table showing the age and gender of subjects who participated in the study to assess thermosensitive delivery platform (TDP) versus liquid control enema.

To compare the subject experiences with each enema group, the questionnaire assessed several issues including the degree of difficulty in retaining the enema, urgency (the need to go to evacuate one's bowels), rectal/abdominal discomfort, flatulence, difficulty with having a bowel movement, and any unpleasant feelings associated with retention, or other adverse events associated with the enema. The results of the survey were assessed in a blinded fashion as well.

FIG. 4 is a graph showing the adverse effects of TDP compared to a liquid control. N=18; mean f SEM; **P<0.01 and ***P<0.001 using two-tailed Student's t-test. Scale: 0—none, 1—slight, 2—moderate, 3—considerable, 4—severe. Overall, there was a statistically significant decrease in issues with retention, urgency, and unpleasant feelings associated with retention of TDP compared to liquid control.

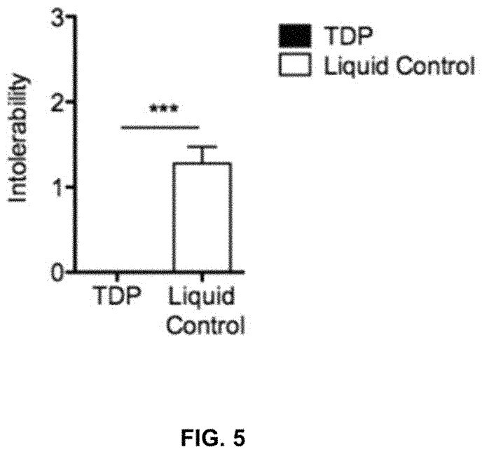

FIG. 5 is a graph showing intolerability of liquid control versus TDP. N=18; mean±SEM; ***P<0.001 using two-tailed Student's t-test. Scale: 0—very tolerable, 1—somewhat tolerable, 2—somewhat intolerable, 3—very intolerable. TDP was found to be much more tolerable when compared to liquid control. All subjects reported TDP to be “very tolerable,” the most favorable measure of tolerability in the survey questionnaire.

After receiving the second enema, subjects were also asked to describe which (if any) enema formulation they preferred. All subjects (N=18) reported preferring the TDP formulation over the liquid control.

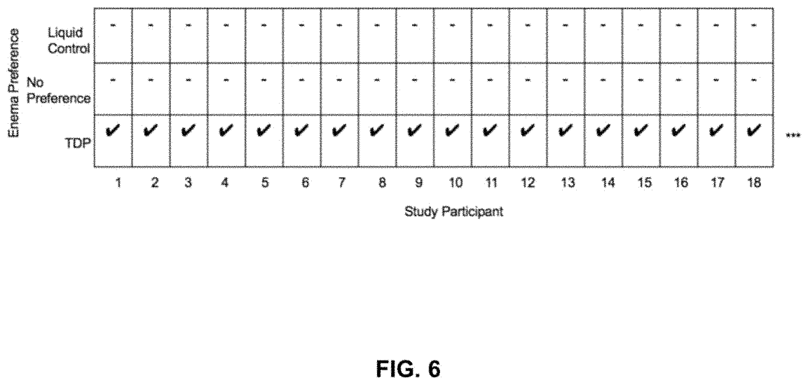

FIG. 6 is a table showing preference of TDP versus liquid control enema. ***P<0.001 using chi-square test.

These results support our hypothesis that TDP could significantly overcome key limitations of topical therapy such as urgency and retention difficulty. There were no adverse outcome/symptom measures where TDP performed worse than liquid control.

Results: TDP Achieves as Good Proximal Distribution as Standard Liquid Enema and Better Retention Over Time

A key advantage of the liquid enema over other forms of topical therapy is the ability of these enemas to reach much further proximally in the colon, potentially treating the entire left colon. To assess extent of proximal distribution, a small amount of iodinated contrast was added to both enemas (TDP and control). X-ray images taken just after enema administration indicated that the maximum proximal distance reached by TDP equaled or exceeded that of the liquid control on average.

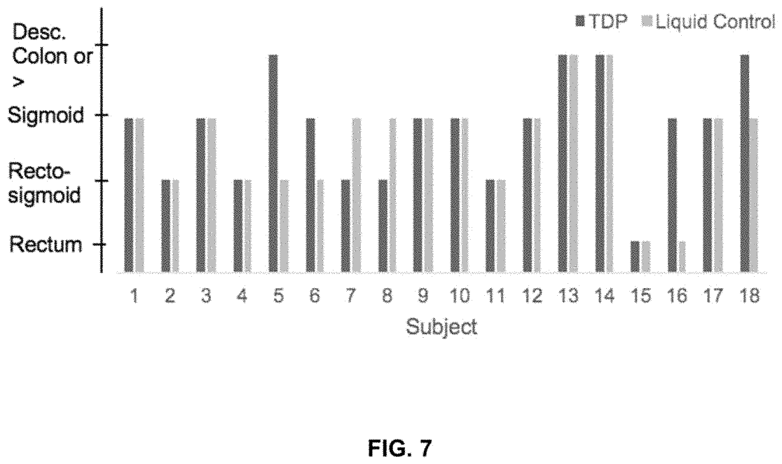

FIG. 7 is a graph showing the maximum proximal distribution of enema after instillation as assessed by x-ray. N=18.

To assess retention of the enema and changes in proximal distribution, repeat x-rays were conducted about 6 hours after the initial TDP or control enema in three subjects (for a total of four x-rays in each of these volunteers). This technique allowed us to assess changes in maximum proximal distribution overtime.

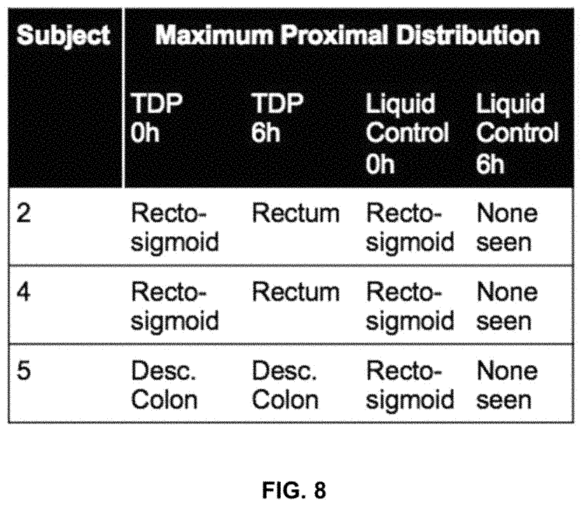

FIG. 8 is a table showing the change in proximal distribution over time of TDP and control enemas.

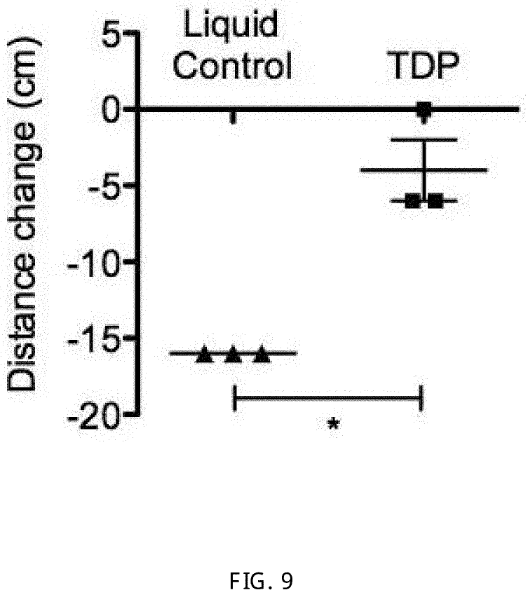

FIG. 9 is a graph showing the change between maximum proximal distance achieved by TDP versus control enema at t=0 hour versus t=6 hour. Change was assessed by serial x-rays; N=3 control and 3 TDP; mean f SEM; *P<0.05 using two-tailed Student's t-test.

In all TDP enemas, TDP remained in the colon at the time of the second x-ray. However, for subjects receiving control enema, no residual enema remained in the colon at the time of the second x-ray. All control enemas originally reached the rectosigmoid colon (about 16 cm from anal verge), but several hours later—at the time of the second x-ray—no enema was detected in the colon, representing an average change of 16 cm. For the TDP, two enemas reached the rectosigmoid colon (about 16 cm from anal verge), and one enema reached the descending colon (about 70 cm from the anal verge). Subjects were allowed to defecate 30 minutes after administration of the enema. At the time of the second x-ray in the subjects given TDP enema, enema was still seen in all three subjects with only a loss of 4 cm.

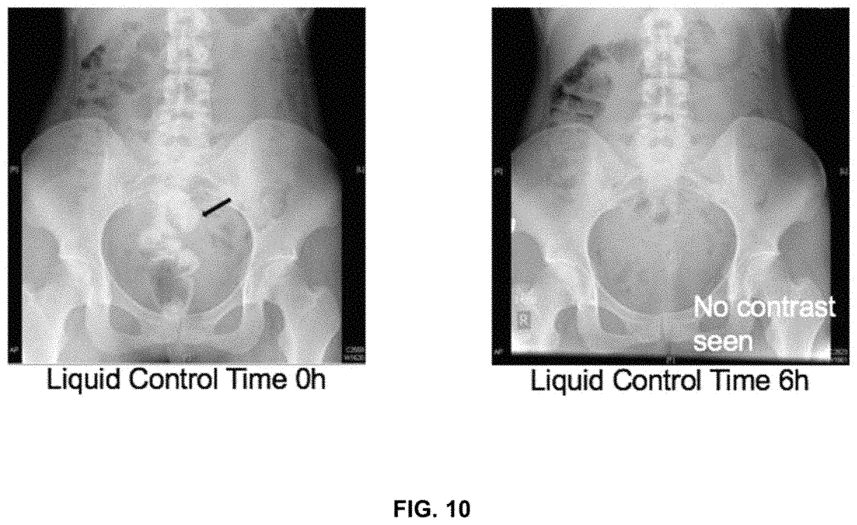

FIG. 10 shows abdominal x-ray images after control enema at t=0 hour versus t=6 hour. Images show maximum retrograde distribution (black arrow; no contrast seen at t=6 hour).

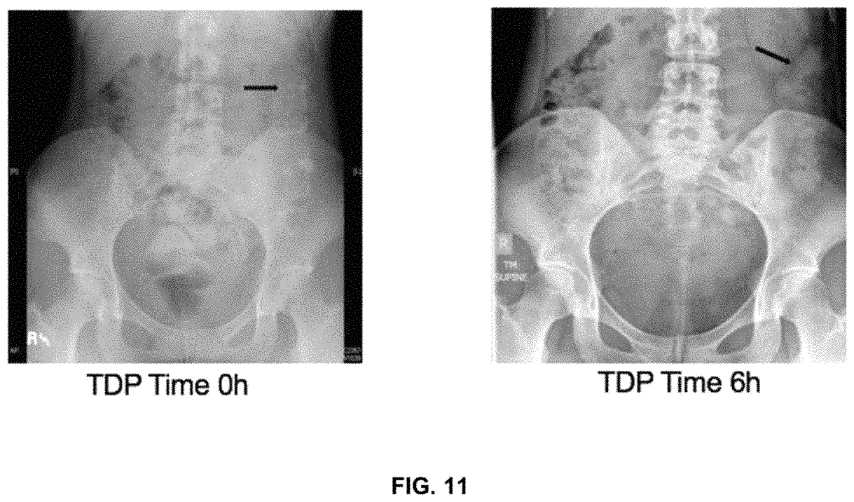

FIG. 11 shows abdominal x-ray images TDP enema at t=0 hour versus t=6 hour. Images show maximum retrograde distribution (black arrows).

Endoscopy (flexible sigmoidoscopy) was performed in two randomly selected subjects within 2 hours of receiving either TDP or control enema and assessed by an endoscopist blinded to the enema type received. In the subject receiving the control enema, the mucosa was visually described as normal in appearance. In the subject receiving TDP, a coating of gel was reported to overlay what was also described as normal colonic mucosa.

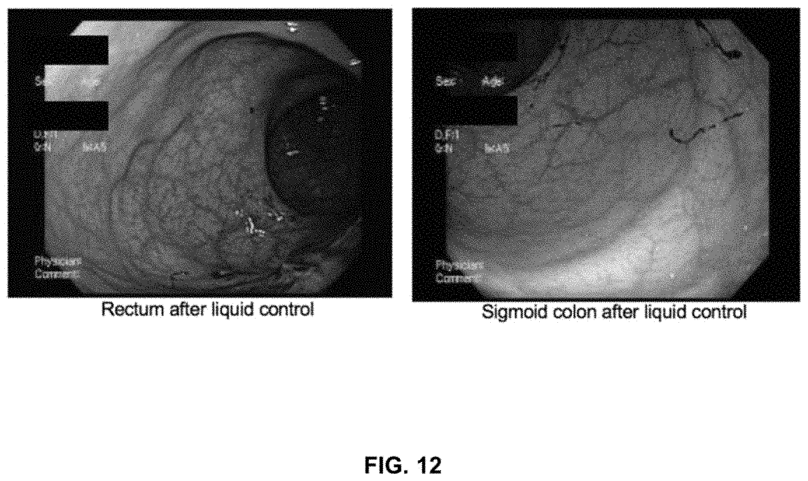

FIG. 12 shows endoscopic images after control enema. Rectum is shown in the left panel, and sigmoid colon is shown in the right panel.

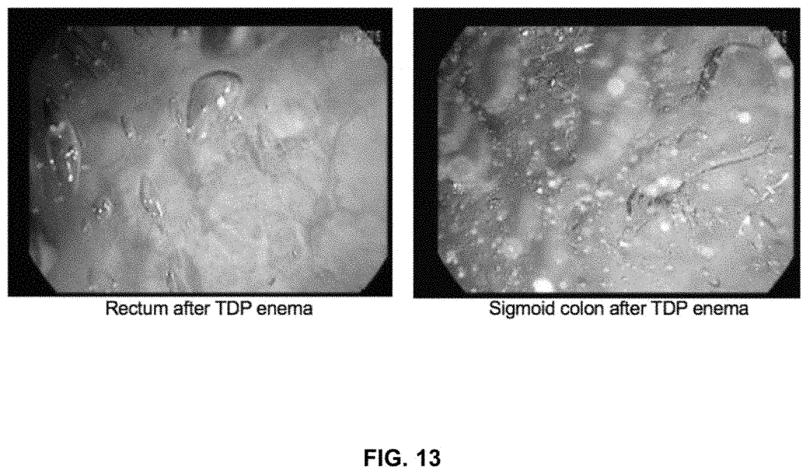

FIG. 13 shows endoscopic images after TDP enema. Rectum is shown in the left panel, and sigmoid colon is shown in the right panel.

Random biopsies of the rectum and sigmoid colon were obtained during both endoscopies. These biopsies were assessed by pathologists blinded to the intervention and all biopsies were described as normal colon tissue on hematoxylin and eosin (H&E) staining.

In addition, plasma samples were taken from two subjects who received TDP enema and one volunteer who had not received enemas. The principal component of TDP, a GRAS polymer, was not detected in any of the plasma samples.

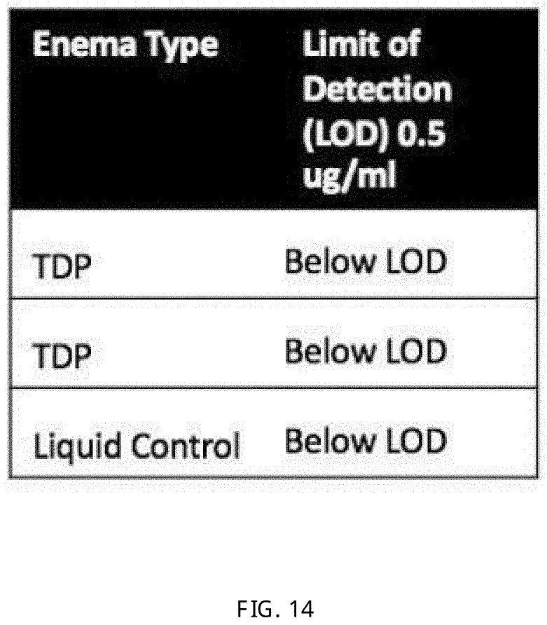

FIG. 14 is a table showing plasma detection of TDP using ultraviolet-visible spectrophotometry. TDP not detected (below limit of detection) in all samples, n=2 TDP, n=1 liquid control.

We further investigated the effect TDP may have on the mucus layer in the colon. We found intact mucus layer in colonic biopsies from both liquid control and TDP, as assessed by staining for the mucin 2 protein component of colonic mucus.

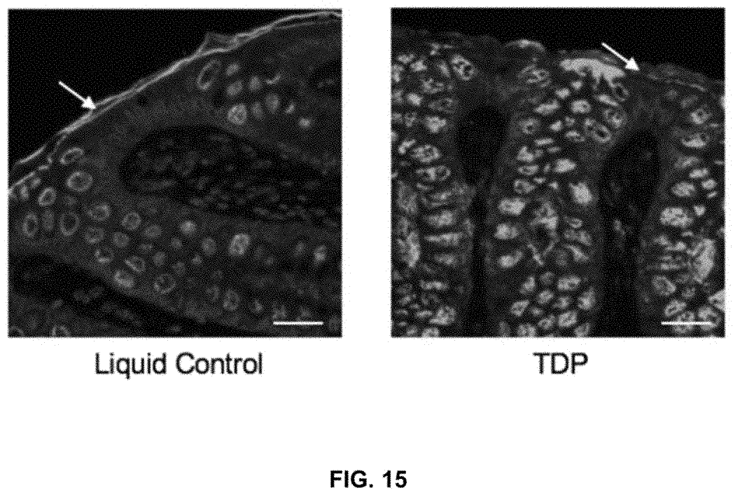

FIG. 15 shows mucus layer staining of colon biopsies. Arrow highlights mucin 2 protein staining. Scale bar is 25 μm.

Discussion

Based on our studies, we have shown TDP overcomes limitations of current topical therapy such as problems with retention and urgency. We have shown that TDP is preferred over traditional liquid enemas and does not cause adverse effects when compared to standard liquid enema. In addition, we have shown that TDP is able to reach as proximally in the colon as liquid enemas. Furthermore, TDP is retained longer compared to liquid control, a finding that is consistent with our work in animal models of colitis. There were no issues with obstruction and no evidence of detectable TDP in the blood following enema administration.

While topical therapy can be used to provide therapy for patients suffering from a range of disorders, including hepatic encephalopathy, constipation, or pain during end of life, they are perhaps most suited for the treatment of IBD. Issues with retention and urgency are perhaps most troubling for patients with colitis, and therefore, TDP with existing anti-inflammatory therapy can potentially better treat these patients. Despite evidence showing that topical therapy has less adverse effects and higher therapeutic efficacy for some types of LBD, topical therapy is still largely underutilized. The issues with existing topical therapy are substantial, as demonstrated by the fact that the majority of patients do not adhere with prescribed therapies.

In addition to overcoming issues with retention and urgency, TDP also has the potential to reduce dosing frequency, given the improved retention and sustained coating of colonic mucosa. While the kinetics of TDP-based treatments needs to be assessed in humans, the ability to reduce the frequency of doses would represent an enormous advance in IBD management. With TDP, we aim to address the key issues such as retention and dosing frequency, which currently prevent widespread adoption of topical treatments for IBD, despite being safe and effective.

To fully understand the potential and possible drawbacks of TDP, further investigation is needed. While the components of TDP are GRAS and used in many over-the-counter products and FDA-approved therapies, including intravenous administration, further studies need to assess the safety of long-term use. Furthermore, while preference of TDP over liquid control is clear in our study of healthy subjects, this needs to be studied in patients with colitis, as they will likely be more sensitive to any rectal instillations due to active disease. The ability of TDP to reduce dosing frequency (likely owing to improved tissue concentrations of therapeutic) in treating clinical colitis is promising and merits further studies. Additionally, given the effectiveness of TDP with approved IBD drugs in treating colitis in animal models, clinical testing will be performed to determine translation to human disease. Overall, TDP represents a unique platform to deliver topical therapies that is better tolerated and preferred over standard liquid enemas and has potential to treat a wide range of diseases.

### CONCLUSIONS

By overcoming issues with current topical therapy, TDP represents a new method to treat colitis that is easier to retain and potentially more effective than current delivery methods. TDP could enable improved targeted delivery of a variety of therapeutics for distal colitis and other disorders.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A method of treating a gastrointestinal condition in a subject, the method comprising:
providing an agent locally to a rectum or colon of a subject having a condition according to a first dosing regimen; and
providing the agent locally to the rectum or colon of the subject according to a second dosing regimen that is different from the first dosing regimen.

2. The method of claim 1, wherein:
the first dosing regimen induces remission of the condition; and
the second dosing regimen maintains remission of the condition.

3. The method of claim 1, wherein each of the first dosing regimen and the second dosing regimen comprises a dosing interval and a dosage of the agent.

4. The method of claim 3, wherein the dosing interval of the first dosing regimen is less than the dosing interval of the second dosing regimen.

5. The method of claim 3, wherein the dosage of the first dosing regimen is greater than the dosage of the second dosing regimen.

6. The method of claim 3, wherein the dosing interval of the first dosing regimen is greater than 12 hours.

7. The method of claim 6, wherein the dosing interval of the first dosing regimen is at least 48 hours.

8. The method of claim 3, wherein the dosing interval of the second dosing regimen is greater than 12 hours.

9. The method of claim 8, wherein the dosing interval of the second dosing regimen is at least 48 hours.

10. The method of claim 1, wherein the agent is provided topically.

11. The method of claim 10, wherein the agent is provided rectally.

12. The method of claim 1, wherein the agent is selected from the group consisting of an aminosalicylate, angiotensin receptor inhibitor, anti-inflammatory, antibiotic, antibody, antimetabolite, antimycotic, antisense oligonucleotide, bacterial sample, beta-blocker, biologic, budesonide, calcineurin inhibitor, corticosteroid, cytokine, growth factor, immunosuppressor, indole, interferon, Janus kinase inhibitor, microbial metabolite, mTOR inhibitor, opioid, PDE4 inhibitor, peptide, pregnane X receptor (PXR) ligand, probiotic, protein, proton pump inhibitor (PPI), salicylic acid derivative, small molecule, sphingosine-1-phosphate receptor modulator, thiopurine, TNF-α binding protein, and toll-like receptor (TLR) ligand.

13. The method of claim 12, wherein the salicylic acid derivative is selected from the group consisting of 5-aminosalicylic acid, 4-aminosalicylic acid, sulfasalazine, olsalazine, and balsalazide.

14. The method of claim 13, wherein the salicylic acid derivative is 5-aminosalicylic acid.

15. The method of claim 1, wherein the agent is provided in a formulation that exists as a liquid at about 23° C. and transitions to a gel at from about 32° C. to about 38° C.

16. The method of claim 15, wherein the formulation comprises the agent in a mixture with a polymer.

17. The method of claim 16, wherein the polymer is a block copolymer comprising polyethylene glycol and polypropylene glycol.

18. The method of claim 15, wherein the formulation comprises at least one lipid.

19. The method of claim 18, wherein the agent is 5-aminosalicylic acid.

20. The method of claim 1, wherein the gastrointestinal condition is selected from the group consisting of Crohn's disease, inflammatory bowel disease, indeterminate colitis, and ulcerative colitis.

21-85. (canceled)
